(19) United States
(12) Patent Application Publication
(10) Pub. No.: US 2024/0215844 A1
(43) Pub. Date: Jul. 4, 2024

(54) Methodology for Assessing Coronary Flow and Microvascular System Through Pressurewires -- Pressure Dilution Curve

(71) Applicant: LightLab Imaging, Inc., Westford, MA (US)

(72) Inventors: Wei C. Chiu, Stony Brook, NY (US); Amir Dolaty, Boxborough, MA (US)

(73) Assignee: Four Robbins Road, Westford, MA (US)

(21) Appl. No.: 18/394,059

(22) Filed: Dec. 22, 2023

Related U.S. Application Data
(60) Provisional application No. 63/477,878, filed on Dec. 30, 2022.

(51) Int. Cl. A61B 5/026; A61B 5/021; A61B 5/0215

(52) U.S. Cl. CPC A61B 5/026; A61B 5/02125; A61B 5/0215; A61B 2562/0247

(57) ABSTRACT

The present disclosure provides systems and methods for determining a mean transit time of blood within a blood vessel using pressure measurements collected by an intravascular tool and not dependent on temperature measurements. The intravascular instrument may collect a plurality of pressure measurements distal to an area of interest within the blood vessel as a bolus passes through the blood vessel and over the instrument. The plurality of pressure measurements may be plotted on a pressure dilution curve. A function of the pressure dilution curve and time is used to determine the mean transit time of the blood within the blood vessel.

### BACKGROUND

Identifying microvascular resistance in a patient may require one or more data collection systems. For example, a physician may use a pressure wire, angiography, intravascular imaging, etc., to collect data to identify microvascular disease. Angiography may provide an insight as to what is happening within an entire heart while a pressure wire may be able to provide certain data measurements within a blood vessel.

Mean transit time, corresponding to the flow rate of blood in a blood vessel is computed by passing a bolus of chilled saline through a blood vessel. The temperature of the bolus is measured as the bolus passes by a proximal and distal temperature sensor on a pressure wire, which is inserted separately from an optical coherence tomography (“OCT”) catheter. A thermodilution curve is then plotted based on the temperature of the bolus as it passes the temperature sensors, providing an indication of the flow rate and, therefore, the mean transit time. This, however, requires additional steps and instruments which can be cumbersome and dangerous in practice.

### BRIEF SUMMARY

The disclosure is generally directed to systems and methods for determining the mean transit time using a pressure dilution curve. The pressure dilution curve may be derived from pressure measurements obtained using an intravascular instrument. For example, the intravascular instrument may be a pressure wire, catheter, or intravascular imaging probe with pressure-sensing capabilities. According to some examples, the intravascular imaging probe may be an OCT probe, micro-OCT probe, near-infrared spectroscopy (“NIRS”) sensor, or an intravascular ultrasound (“IVUS”) probe. The mean transit time may be the rate of flow within the blood vessel.

The intravascular instrument may be inserted into the blood vessel at a location distal to an area of interest. The intravascular instrument may have a distal aperture connected to an external pressure transducer The intravascular instrument may have a column of an incompressible fluid, such as contrast, saline, or any suitable hemocompatible fluid. The intravascular instrument may collect a plurality of pressure readings at the distal aperture as the intravascular instrument is advanced through the blood vessel to the area of interest. The system may use the pressure readings to determine and/or create a pressure dilution curve. The pressure dilution curve may be used to determine the mean transit time within the blood vessel. In some examples, the pressure dilution curve may allow for other downstream flow indicators, such as coronary flow reserve (“CFR”), index of microcirculatory resistance (“IMR”), etc.

One aspect of the disclosure includes a method comprising receiving, at one to more processors, intravascular data for a blood vessel, the intravascular data comprising a plurality of pressure measurements at a location within the blood vessel collected by an intravascular instrument, and determining, by one or more processors based on the plurality of pressure measurements, a mean transit time of blood within the blood vessel, wherein the determining is not dependent on blood vessel temperature measurements.

The method may further comprise determining the mean transit time by determining, by one or more of the processors, a ratio of an integral of a given average pressure measurement multiplied by a time given average pressure measurement to an integral of the given average pressure measurement.

The method may further comprise determining, by one or more processors based on the plurality of pressure measurements, an average pressure measurement and creating, by one or more processors based on the determined average pressure measurement, a distribution curve.

The method may further comprise providing for display one or more indicators of the blood flow based on the determined mean transit time, wherein the indicators of blood flow may comprise at least one of a value, color, graphic, or animation.

The method may further comprise determining the mean transit time of the blood within the blood vessel based on the distribution curve.

The method may further comprise determining the mean transit time, by the one or more processors, using a ratio of an integral of a given average pressure measurement multiplied by a time of the given average pressure measurement to an integral of the given average pressure measurement.

The method may further comprise one or more processors configured to determine, based on the determined mean transit time, at least one of a coronary flow reserve (“CFR”) value or an index of microvascular resistance (“IMR”) value.

The method wherein when determining the average pressure measurement, the one or more processors are further configured to use a plurality of cycles of the plurality of intravascular data from including the pressure readings before during, and after the intravascular instrument reaches a location in the blood vessel.

The method may further comprise determining, by one or more processors, the averaged pressure waveform by identifying a first pressure measurement of the plurality of pressure measurements as the intravascular instrument advances to the area of interest.

The method may further comprise the intravascular instrument is one of a pressure wire or an intravascular imaging tool or a catheter. The intravascular imaging tool may be an optical coherence tomography (“OCT”) probe or an intravascular ultrasound (“IVUS”) probe.

The intravascular instrument may have pressure-sensing capabilities with a pressure transducer coupled to a pressure-assessing port.

Another aspect of the disclosure includes a system comprising one or more processors configured to receive a plurality of pressure measurements at a location within the blood vessel. The one or more processors may determine, based on the plurality of pressure measurements, a mean transit time of blood within the blood vessel, wherein the determining is not dependent on blood vessel temperature measurements.

The system may further comprise an intravascular instrument in communication with the one or more processors configured to collect the plurality of pressure measurements.

The one or more processors may be further configured to determine, based on the mean transit time, at least one of a CFR value or IMR value.

The one or more processors may be further configured to determine average pressure measurements and create, based on the determined average pressure measurements, a distribution curve. Moreover, the one or more processors may determine the mean transit time of the blood within the blood vessel based on the distribution curve.

The system may further comprise one or more of the processors determining the mean transit time by determining, by the one or more processors, a ratio of an integral of a given average pressure measurement multiplied by a time of the given pressure measurement to an integral of the given average pressure measurement.

The one or more processors may be configured to use a plurality of cycles of the plurality of the intravascular data from including the pressure readings before during and after the intravascular instrument reaches a location in the blood vessel. The one or more processors may be configured to integrate the distribution curve when determining the mean transit time.

The intravascular instrument may be one of a pressure wire, a catheter, or an intravascular imaging tool with pressure-sensing capabilities. The intravascular imaging tool may be an OCT or IVUS probe with a purge port.

The system may further comprise the intravascular instrument collecting a plurality of pressure measurements distal to the area of interest in the blood vessel.

The system may further comprise a column of fluid within the intravascular device, wherein the fluid may be either saline, contrast, or hemocompatible fluid.

Another aspect of the disclosure related to a pressure-sensing catheter comprising a hypotube configured to deliver a hemocompatible fluid to a target area of a blood vessel, an outer member having a proximal end and a distal end, wherein the proximal end is secured to the hypotube, an inner member having a proximal end and a distal end, wherein the proximal end of the inner tube is secured to the outer member, and at least one aperture on the distal end of the outer member, the aperture allowing for measurement of pressure at the location within the blood vessel.

The pressure sensing catheter may further comprise one or more processors coupled to the pressure sensing catheter configured to receive pressure measurements and determine, by the one or more processors based on the plurality of pressure measurements, a mean transit time of blood within the blood vessel, wherein the determining is not dependent on temperature measurements.

The pressure-sensing catheter may be coupled to a pressure transducer in fluid communication with at least one of the apertures on the distal end of the outer member.

The pressure-sensing catheter may further be coupled to one or more processors that are configured to determine, based on the determined mean transit time, at least one of a CFR value or an IMR value.

The pressure-sensing catheter may further be coupled to one or more processors that are configured to determine average pressure measurements and create, based on the determined average pressure measurements a distribution curve. One or more processors may determine the mean transit time of the blood within the blood vessel based on the distribution curve.

The pressure-sensing catheter may further be coupled to one or more processors configured to determine a ratio of an integral of a given average pressure measurement multiplied by a time of the given average pressure measurement to an integral of the given average pressure measurement.

The pressure-sensing catheter may be coupled to one or more processors further configured to determine the average pressure measurement by using a plurality of cycles of the plurality of the intravascular data from including the pressure readings before during and after the intravascular instrument reaches a location in the blood vessel.

The pressure-sensing catheter may be coupled to one or more processors that are further configured to integrate the distribution curve when the mean transit time.

The pressure-sensing catheter may collect a plurality of pressure measurements distal to the area of interest.

The method may further comprise determining, by one or more processors, the mean transit time using a value of an area defined by the lowest point of the distribution curve.

### BRIEF DESCRIPTION OF THE DRAWINGS

FIG. 1 is an example system including a pressure wire according to aspects of the disclosure.

FIG. 2 is an illustration of an experimental setup according to aspects of the disclosure.

FIG. 3 is an example system including an intravascular imaging probe according to aspects of the disclosure.

FIG. 4 is an illustration of an experimental setup according to aspects of the disclosure.

FIG. 5 is an example system with a pressure-sensing catheter according to aspects of the disclosure.

FIGS. 6A and 6B cross-sectional views of the distal end of the pressure sensing catheter.

FIG. 7 is an example system with an OCT catheter according to aspects of the disclosure.

FIG. 8 is an example system with a delivery catheter according to aspects of the disclosure.

FIG. 9 is an example averaged pressure waveform from raw pressure sensor measurements.

FIG. 10A is an example of pressure dilution curve according to aspects of the disclosure and an example of a functional representation of mean transit time according to aspects of the disclosure.

FIG. 10B is another example of pressure dilution curve according to aspects of the disclosure and an example of a functional representation of mean transit time according to aspects of the disclosure.

FIG. 11A is another example of a pressure dilution curve according to aspects of the disclosure and

FIG. 11B is an example of a functional representation of mean transit time according to aspects of the disclosure.

FIG. 12A is a scatter plot of experimental results of determining mean transit time from thermodilution method and true flow rate.

FIG. 12B is a scatter plot of experimental results of determining mean transit time from thermodilution and area-under-the curve methods.

FIG. 12C is a scatter plot of experimental results of determining mean transit time from area-under-the-curve method and true flow rate.

FIG. 13A is a flow diagram illustrating a method of determining mean transit time of blood within a blood vessel according to aspects of the disclosure.

FIG. 13B is a flow diagram illustrating an alternative method of determining mean transit time of blood within a blood vessel according to aspects of the disclosure.

### DETAILED DESCRIPTION

The technology provides for using a single intravascular instrument to identify and/or diagnose one or more aspects of microvascular disease within a blood vessel. For example, blood flow may be determined using only pressure measurements, without temperature measurements as required by existing systems. By using a single intravascular instrument, the risk to the patient during the procedure decreases, as there may be fewer incisions, fewer instruments inserted into the patient, less time in the procedure/operating room, etc. In some examples, by using a single intravascular instrument, diagnosing microvascular disease in a patient may be performed more efficiently because a physician is able to collect more data at once.

According to some examples, an intravascular instrument may be used to collect intravascular data of a blood vessel. The intravascular data may be, for example, pressure measurements. The intravascular data may be used to determine the mean transit time of blood within the blood vessel. In some examples, the mean transit time may be determined at rest and/or at hyperemia. The mean transmit time determined from the pressure measurements may be used to determine coronary flow reserve (“CFR”), index of microcirculatory resistance (“IMR”), or other diagnostic indices. The CFR and/or IMR values may be used to diagnose microvascular disease. According to some examples, pressure measurements and/or any values determined using the pressure measurements may be used to evaluate a lesion within a blood vessel, evaluate potential stent placement, diagnose microvascular disease, etc.

According to some examples, additional factors, combined with the determined CFR and/or IMR, may be used to identify one or more aspects of microvascular disease within the blood vessel. The additional factors may include, for example, the age, gender, body mass index (“BMI”), medical history, vessel type, vessel condition, treatment history, etc. of the patient. The medical history may include, for example, known heart failure, a previous diagnosis of diabetes, hypertension, etc. Vessel types may include the left anterior descending (“LAD”) artery, left circumflex artery (“LCX”), right coronary artery (“RCA”), left marginal artery, diagonal arteries, right marginal artery, etc. Treatment history may include, for example, prior percutaneous coronary intervention (“PCI”), coronary artery bypass graft (“CABG”), etc.

By using pressure measurements, as an alternative to temperature measurements, the system and software used to calculate mean transit time may be operated without strict adherence to parameters regarding the methodology of injecting the bolus. Accordingly, errors may be reduced that might otherwise result from the injection of the bolus at an imperfect time or temperature. Additionally, the intravascular instrument will not need temperature sensing capabilities, thereby reducing the cost and complexity of the required instrumentation. For example, the mean transit time may be determined by using a pressure transducer coupled to the purge port of the OCT catheter to collect pressure measurements, and using the pressure measurements to determine the mean transit time may eliminate the need to insert a pressure wire. This may reduce the risk of complications during the procedure. Additionally or alternatively, using a catheter to determine the mean transit time may allow for real-time readings and computation of the mean transit time. In some examples, the mean transit time, determined using the pressure dilution curve, may be used to determine CFR data and/or determine IMR data. Thus, the pressure dilution curve may be used to determine physiological information as well as anatomical information.

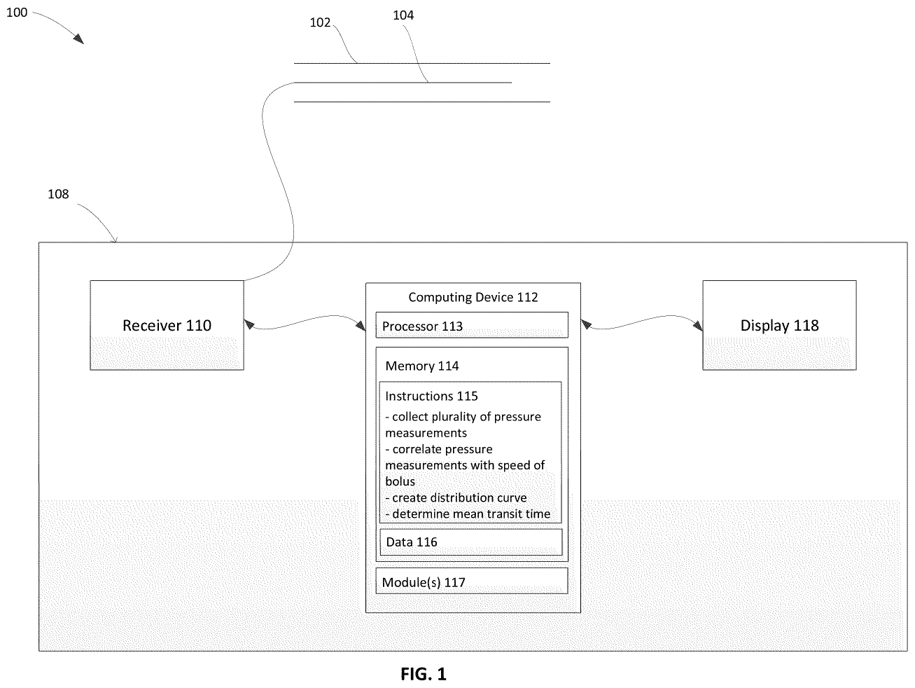

FIG. 1 illustrates a data collection system 100 for use in collecting intravascular data. The system may include an intravascular instrument that can be used to collect data within a blood vessel 102. The intravascular instrument may be a pressure wire, a pressure sensing catheter, an OCT probe, a micro-OCT probe, near a NIRS sensor, or an IVUS catheter. While FIG. 1 illustrates the use of a pressure wire, the use of a pressure wire is not intended to be limiting. An IVUS catheter, NIRS sensor, OCT probe, etc. may be used in conjunction with or instead of the pressure wire, described in detail below. A guide catheter, not shown, may be used to introduce the pressure wire 104 into the blood vessel 102. The pressure wire 104 may be introduced and positioned distal to an area of interest within the blood vessel 104. In some examples, the area of interest may be a location in the blood vessel where there is a high calcium or plaque buildup that has restricted blood flow or a possible location for a stent.

According to some examples, the pressure wire 104 may be introduced and held stationary such that a plurality of intravascular measurements may be captured. The pressure wire 104 may held stationary distal to an area of interest, such that distal pressure measurements are captured. A bolus may be injected into blood vessel 102 through a purge port at a location such that the bolus will flow over the pressure wire 104. The pressure wire 104 may capture intravascular measurements as the bolus passes. According to some examples, the bolus of saline may be room temperature or consistent with the body temperature of the patient. The intravascular measurements captured by the pressure wire 104 may be used to identify features of the blood vessel 102.

The pressure wire 104 may be communicatively connected to subsystem 108 via a wired or wireless connection. The subsystem 108 may include a receiver 110 which transmits a signal to a computing device 112. The computing device 112 may include one or more processors 113, memory 114, instructions 115, data 116, and one or more modules 117.

The one or more processors 113 may be any conventional processors, such as commercially available microprocessors. Alternatively, the one or more processors may be a dedicated device such as an application specific integrated circuit (ASIC) or other hardware-based processor. Although FIG. 1 functionally illustrates the processor, memory, and other elements of device 110 as being within the same block, it will be understood by those of ordinary skill in the art that the processor, computing device, or memory may actually include multiple processors, computing devices, or memories that may or may not be stored within the same physical housing. Similarly, the memory may be a hard drive or other storage media located in a housing different from that of device 112. Accordingly, references to a processor or computing device will be understood to include references to a collection of processors or computing devices or memories that may or may not operate in parallel. By way of example, a processor within the computing device 112 may be communicatively coupled with a remote processor, such as through a wired or wireless network, such as the Internet. The remote processor may perform some or all of the computing and coordinate with the computing device 112.

Memory 114 may store information that is accessible by the processors, including instructions 115 which may be executed by the processors 113, and data 116. The memory 114 may be a type of memory operative to store information accessible by the processors 113, including a non-transitory computer-readable medium, or other medium that stores data that may be read with the aid of an electronic device, such as a hard-drive, memory card, read-only memory (“ROM”), random access memory (“RAM”), optical disks, as well as other write-capable and read-only memories. The subject matter disclosed herein may include different combinations of the foregoing, whereby different portions of instructions 115 and data 116 are stored on different types of media.

Memory 114 may be retrieved, stored, or modified by processors 113 in accordance with instructions 115. For instance, although the present disclosure is not limited by a particular data structure, the data 116 may be stored in computer registers, in a relational database as a table having a plurality of different fields and records, XML documents, or flat files. The data 116 may also be formatted in a computer-readable format such as, but not limited to, binary values, ASCII, or Unicode. By further way of example only, the data 116 may be stored as bitmaps comprised of pixels that are stored in compressed or uncompressed, or computer instructions for drawing graphics. Moreover, the data 116 may comprise information sufficient to identify the relevant information, such as numbers, descriptive text, proprietary codes, pointers, references to data stored in other memories (including other network locations), or information that is used by a function to calculate the relevant data.

The instructions 115 can be any set of instructions to be executed directly, such as machine code, or indirectly, such as scripts, by the processor 113. In that regard, the terms “instructions,” “application,” “steps,” and “programs” can be used interchangeably herein. The instructions can be stored in object code format for direct processing by the processor, or in any other computing device language including scripts or collections of independent source code modules that are interpreted on demand or compiled in advance. The functions, methods, and routines of the instructions are explained in more detail below.

Modules 117 may include a plaque, such as calcium plaque, a detection module, a display module, a flow rate or mean transit time module, a pressure change module, a temperature change module, stent detection, or other detection and display modules. For example, the computing device 112 may access a flow rate module for detecting the mean transit time of blood in the blood vessel.

Subsystem 108 may include a display 118 for outputting content to an operator. As shown, display 118 is separate from computing device 112. However, according to some examples, display 118 may be part of computing device 112. Display 118 may output pressure data relating to one or more features detected in the blood vessel. For example, the output may include, without limitation, pressure data, thermodilution data, aortic and distal pressure, pressure-based indicia of risk posed to stent expansion, flow rate, etc. Display 118 may identify features with a value, text, arrows, color coding, highlighting, contour lines, animation, or other suitable human or machine-readable indicia. For example, the display may output green if the pressure of the blood within the blood vessel is within the given range of pressure measurements and red if the pressure is outside of that range. The range may be programmed by the operator, preset within the processors, or configured by artificial intelligence within the processors. The display may output a graphic that may be an icon, picture, curve, or other visual image. The display may output an animation that may be a depiction of the blood vessel of the patient with an animation of blood flowing through at a rate of speed corresponding to the determined mean transit time.

According to some examples, the display 118 may include a graphical user interface (“GUI”). One or more steps may be performed automatically or without user input to navigate images, input information, select and/or interact with an input, etc. Display 118 alone or in combination with computing device 112 may allow for toggling between one or more viewing modes in response to user inputs. For example, a user may be able to toggle between different side branches on display 118, such as by selecting a particular side branch and/or by selecting a view associated with the particular side branch.

In some examples, the display 118, alone or in combination with computing device 112, may include a menu. The menu may allow a user to show or hide various features. There may be more than one menu. For example, there may be a menu for selecting blood vessel features to display. Additionally or alternatively, there may be a menu for selecting the target area of the blood vessel where the pressure measurements may be collected.

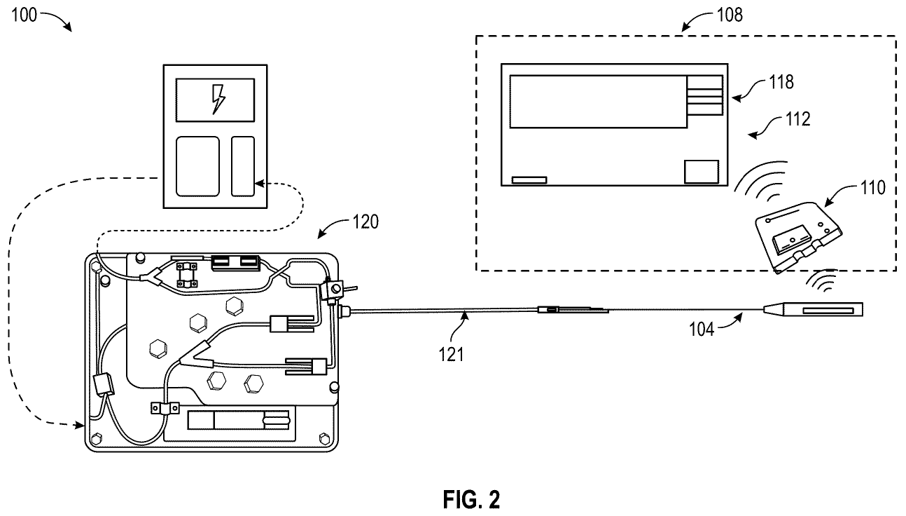

FIG. 2 illustrates a system for data collection system 100. System 100 includes pressure wire 104, subsystem 108, including receiver 110, computing device 112, and display 118, physiology phantom 120, and guide catheter 121. The system as depicted may simulate a clinical scenario for collecting pressure measurements using an intravascular instrument.

The physiology phantom 120 can simulate vascular physiology and represents the human vascular system. The physiology phantom 120 is a combination of hardware and software used to mimic the heart flow of a patient for a benchtop experiment. The hardware of the physiology phantom represents a branch of the coronary artery. The software of the physiology phantom 120 controls the internal hardware, generating conditions found within a coronary artery, such as waveforms similar to the beat of a heart.

The guide catheter 121 may be used to introduce the pressure wire 104 into the blood vessel 102. The guide catheter 121 may be any tube-like delivery apparatus such as any catheter, a guiding sheath, a trocar, or a tubular instrument, or the like capable of being used for percutaneous intracoronary (PCI) procedures. Additionally, the guide catheter 121 may measure the aortic pressure of the blood vessel. The guide catheter 121 may be inserted into the blood vessel, hyperemia may be pharmacologically induced using adenosine, and the aortic pressure at the opening of the blood vessel may be measured. In some examples, the guide catheter 121 may be connected to a separate receiver via a wired or wireless connection. The receiver may collect the aortic pressure readings and transmit them to subsystem 108 to be used for intravascular calculations.

According to some examples, pressure wire 104 may be inserted into physiology phantom 120 through a guide catheter 121. The guide catheter 121 may be inserted into the physiology phantom 120. The pressure wire 104 may be inserted into the guide catheter 121 into the blood vessel 102 and advanced to the area of interest. The pressure wire 104 may communicate with the receiver 110 via a wired or wireless connection. In examples where the connection is a wireless connection, the pressure wire 104 and receiver 110 may communicate via Bluetooth, near-field communication (“NFC”), Wi-Fi, or other short-range communications interfaces.

According to some examples, subsystem 108 may include components within a single housing components outside of the housing, or in multiple housings. As illustrated, the receiver 110 is in a separate housing and wirelessly connected to a single housing that contains the computing device 112 and display 118, as described above.

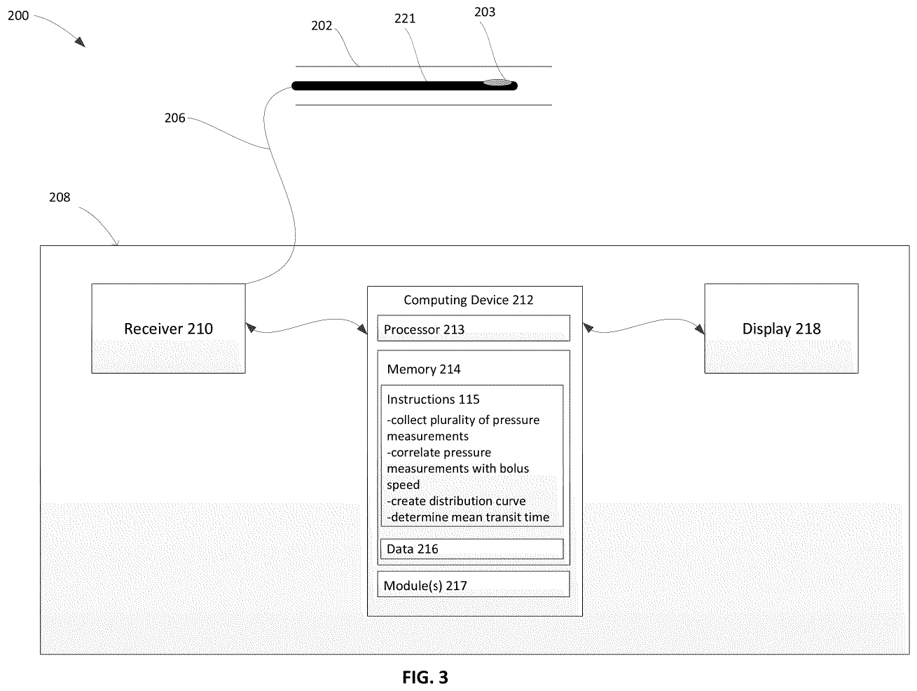

FIG. 3 illustrates another data collection system 200 for collecting intravascular pressure data. System 200 may operate the same or substantially the same as system 100. The components of system 200 may be similar to system 100, except the intravascular instrument of system 200 is a pressure sensing catheter 221 instead of a pressure wire 104. The system may be coupled with an intravascular imaging probe, such as an OCT probe, micro-OCT, NIRS sensor, or an intravascular ultrasound (“IVUS”) probe. The catheter 221 may be equipped with a distal aperture 203. In some examples, the distal aperture 203 may be within a catheter and coupled to an external pressure transducer. In some examples, the pressure transducer may be coupled to the pressure assessing port of the catheter 221.

While only one distal aperture 203 is depicted, this is not intended to limit the number of distal apertures 203 on the catheter 221. In an example, the distal aperture 203 may be positioned at the distal end of the catheter. Additionally or alternatively, one or more apertures may be positioned near the mid-section of the catheter. Additionally or alternatively, one or more apertures may be positioned near the proximal end of the catheter. In yet another example, there may be one or more apertures anywhere on the body of the catheter, such as one or more apertures on the distal end of the catheter as well as one or more apertures on the mid-section of the catheter.

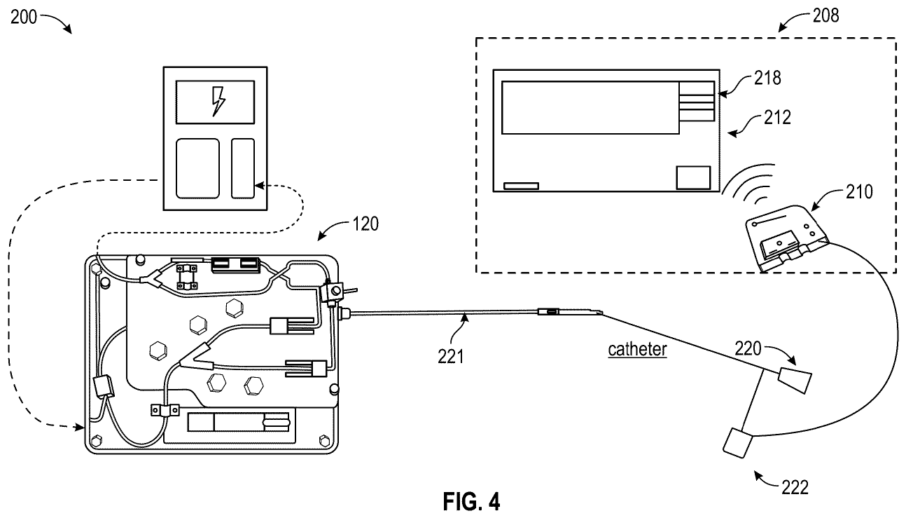

FIG. 4 illustrates the benchtop setup of the data collection system with an intravascular imaging tool, similar to the system described in FIG. 3. System 200 includes pressure assessing port 220 of the imaging tool connected to pressure transducer 222 to subsystem 208, including receiver 210, computing device 212, and display 218, and to distal aperture 203, not shown. The catheter 221 is inserted into the physiology phantom 120. The system as depicted may simulate a clinical scenario for collecting pressure measurements using an intravascular instrument.

The distal aperture may be in fluid communication with the pressure transducer 222. The pressure transducer 222 may be coupled to the pressure assessing port 220 at the end of the catheter 221. The pressure transducer 222 may be communicatively connected to the receiver 210 via a wired or wireless connection to collect a plurality of pressure measurements. The catheter 221 may be filled with an incompressible fluid before it is inserted into the blood vessel. The incompressible fluid may consist of contrast, saline, hemocompatible fluid, or another medium. The pressure transducer 222 may measure the hydraulic column pressure of the catheter 221 as it is inserted into the blood vessel.

For example, the distal aperture may be positioned distal to the area of interest. The hydraulic column pressure of the catheter may vary based on the physiology of the blood vessel where the distal aperture is located, specifically an increase or decrease in the blood pressure where the vessel narrows or widens. The pressure transducer 222 may receive the pressure readings of the blood vessel at the distal aperture as it is inserted into the blood vessel. The user may select an area of interest for the system to use for calculations.

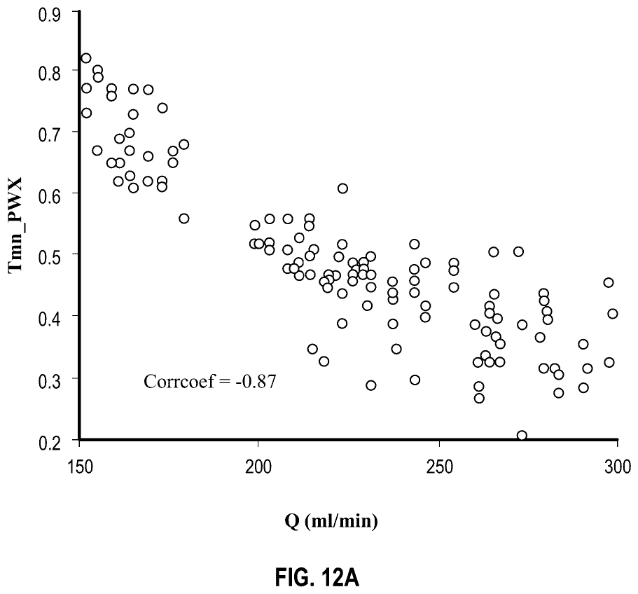

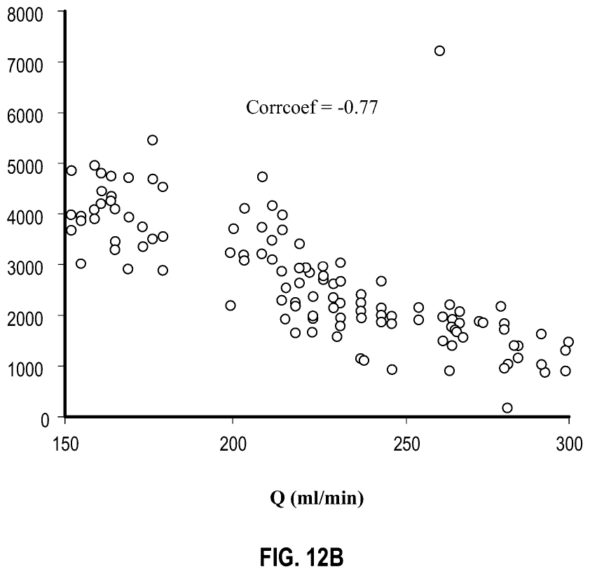

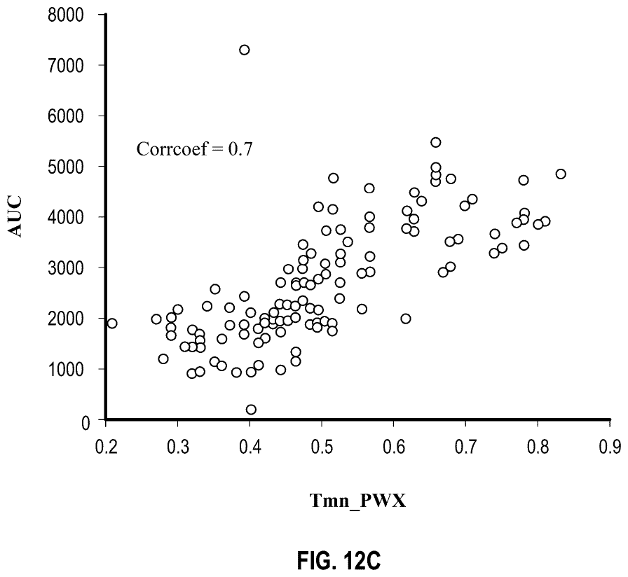

The sequence of pressure measurements may show the blood vessel before, during, and after the distal aperture reaches the area of interest. The sequence of pressure measurements may be used to determine the flow rate of the blood within the blood vessel. For example, each pressure measurement may include a timestamp to indicate when the pressure measurement was taken at the distal aperture 203. In some examples, the pressure measurements may be taken at a predetermined interval of time and marked as such. For example, the system may record pressure measurements every 0.01 seconds as the catheter is advanced to the area of interest. The pressure within the blood vessel 202 may be determined. The determined pressure measurement of the blood vessel where the distal aperture 203 is located may be used, along with the time stamp or time interval, to determine the flow, and, therefore, the mean transit time of blood within the blood vessel. As described more fully below with respect to FIG. 12, the mean transit time may be determined based on a ratio of an integral of the average pressure measurement at a given time multiplied by the time to the integral of the average pressure measurement at that time.

In this example, the pressure transducer 222 may be communicatively connected to subsystem 208 via a wireless or wired connection 206. The subsystem 208 may include a receiver 110 which transmits the pressure signal to a computing device 212.

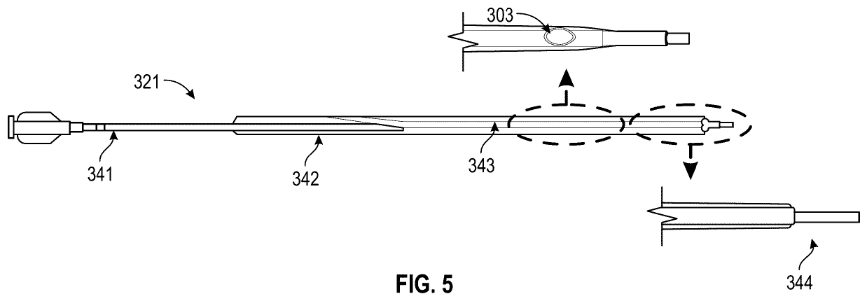

FIG. 5 illustrates an example system employing a catheter configured to measure pressure variations within the blood vessel. The catheter 321 may be any catheter that may be connected to a pressure transducer.

The catheter 321 may comprise a proximal end having a side arm and a hypotube 341. The side arm and the hypotube 341 may facilitate the delivery of contrast media, saline, or a hemocompatible fluid, to the target area of the blood vessel. The catheter 321 may further comprise a distal end having two polymeric members. The two polymeric members may be an outer member 342 and an inner member 343. The outer member 342 may be fixed to the hypotube 341, such as by being bonded directly to the hypotube 341. The inner member 343 may be joined to the outer member 342 to form a rapid exchange (RX) notch. In some embodiments, the catheter may be configured as an over-the-wire catheter with a side arm. The inner member 343 may provide a means for delivering the catheter 321 over a guide wire 344. The space between the outer member 342 and the inner member 343 may provide a space for a column of incompressible fluid, such as contrast, saline, or a hemocompatible fluid, to the target vessel. The column of fluid may enable the direct pressure in the blood vessel to be measured at the distal aperture 303 of the catheter 321.

In some examples the catheter 321 may comprise distal apertures on the distal end of the catheter, such as distal aperture 303. The distal aperture 303 may be positioned on the distal end of the catheter 321. At the location of the distal aperture 303, the column of fluid is subjected to variations of the vessel, such as variations in the blood pressure. In some embodiments, the distal aperture 303 may capture the variations in the vessel as vessel data and transmit the vessel data through the catheter 321 to a pressure transducer. Once the vessel data is received at the distal aperture 303, the pressure transducer may collect direct pressure measurements.

In some examples, the distal aperture 303 may be on the distal end of the catheter 321. The distal aperture 303 may be positioned perpendicular to the flow of the blood in the vessel. Alternatively, the distal aperture 303 may be on the outer surface of the outer member 342, and parallel to the flow path.

While only one distal aperture 303 is depicted, this is not intended to limit the number of distal apertures coupled to and/or integral with catheter 321. In some examples, multiple apertures are distributed circumferentially around the outer member. In another example, the apertures may be distributed laterally along one or more portions of the outer member.

The apertures may be formed in the outer member 342 using a heat process. The apertures may be formed with specialized contouring around the edges of the aperture. The contouring may prevent fluid turbulence as the blood passes over the aperture.

The apertures may have varying sizes between, 0.01 mm²-4 mm². In some examples, the apertures have varying sizes between 0.025 mm²-2 mm². The apertures may vary in shape, such as circular, square, triangular, or any polygonal shape.

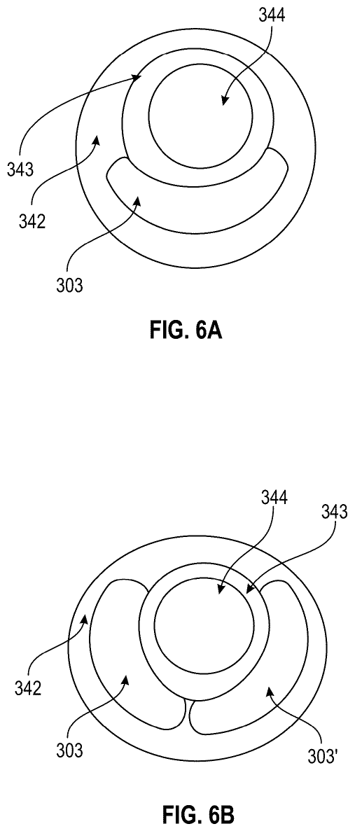

FIG. 6A illustrates a cross-sectional view of the distal end of the catheter 321 described in FIG. 5. The distal end of the catheter 321 may include the outer member 342 and the inner member 343. The outer member 342 may feature a distal aperture 303. The inner member may provide a path for the guide wire 344 to run through for proper placement of the catheter 321 into the blood vessel.

FIG. 6B illustrates another cross-sectional view of the distal end of the catheter, in which there are two distal apertures. The distal end of the catheter 321 may include the outer member 342 and the inner member 343. The outer member 342 may feature two distal apertures 303 and 303′. Multiple distal apertures may provide for more accurate blood vessel pressure measurements. The inner member may provide a path for the guide wire 344 to run through for proper placement of the catheter 321 into the blood vessel.

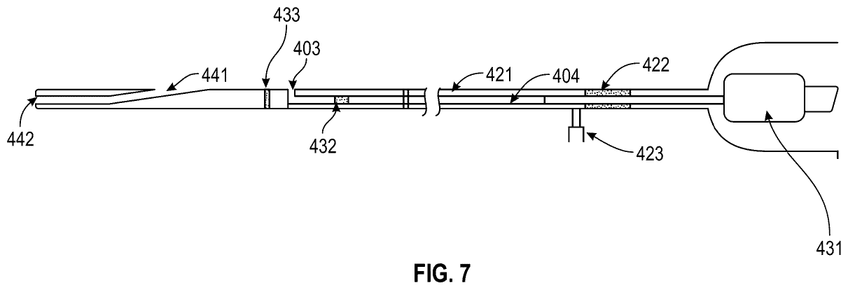

FIG. 7 illustrates a catheter similar to that described in FIG. 5 but equipped with an imaging tool. The catheter 421 may have at least one distal aperture 403, a liquid seal 422, and a purge port 423. The catheter 421 may also have a guidewire entry point 441 and a guidewire exit 442. In this example, the catheter 421 has an imaging tool 404 that is an OCT probe, thus it may have a fiber optic connector 431, an OCT lens 432, and a radio-opaque marker 433. In other examples, the imaging tool 404 may be an IVUS probe, NIRS probe, micro-OCT probe, etc.

In this example, the purge port 423 takes the place of the pressure assessing port, as described in FIG. 4. The distal aperture may be coupled to the purge port 423 via a pressure transducer. The pressure transducer may be communicatively connected to the receiver via a wired or wireless connection to collect a plurality of pressure measurements. According to methods known in the art, the pressure proximal to the area of interest, also understood as the aortic pressure, may be measured by the guide catheter.

The catheter may hold a column of incompressible fluid to facilitate pressure measurements at the location of the distal aperture 403. In this example, the incompressible fluid may be any hemocompatible fluid that will not interfere with the imaging tool, such as saline or another suitable medium. The column of fluid is held in place by the liquid seal 422. The liquid seal 422 prevents the bolus away from the distal end of the catheter 421. The column of fluid is held around the imaging tool 404. The purge port 423 may be used to push a bolus of fluid to areas of interest to properly image the area. The purge port 423 may be used to fill the catheter 421 with the column of fluid for the pressure measurements.

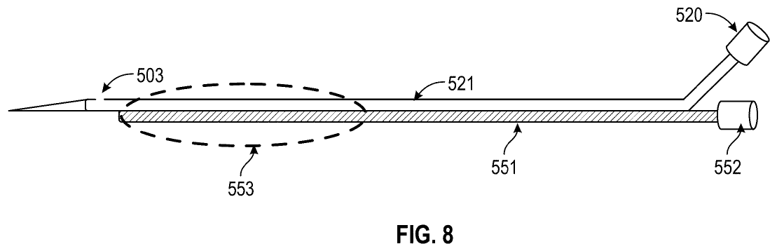

The distal aperture 403 may be positioned distal to the area of interest, such that the pressure transducer measures pressure distal to the area of interest at the distal aperture 403. The measurement is initiated when the distal aperture reaches the area of interest. As the distal aperture 403 approaches the area of interest, the pressure transducer may, either continuously or at a high rate of acquisition, collect a plurality of distal pressure measurements of the area of interest. The pressure measurements may be taken by the pressure transducer at the distal aperture within the blood vessel before, during, and after the distal aperture reaches the area of interest. The pressure measurements can be expressed as a function of time and provide a profile of the pressure across the area of interest. This function, as explained in more detail below regarding FIGS. 8-10, is used to determine the mean transit time of the blood. The pressure measurements may be used to determine the flow rate of the blood within the blood vessel. For example, each pressure measurement may be assigned a corresponding timestamp based on where the distal aperture 403 was located when the pressure measurement was taken, such as before, during, and after the distal aperture reaches the area of interest. In some examples, the pressure measurements may be taken at a predetermined interval of time and marked as such. For example, the system may record pressure measurements every 0.01 seconds as the catheter is advanced to the area of interest. The pressure measurement of the blood vessel at the distal aperture 403 may be used to determine the flow and, therefore, the mean transit time of blood within the blood vessel. Additionally, the raw pressure waveform might be utilized to derive coronary indices, such as Resting Full-Cycle Ratio (RFR) and Instantaneous wave-free Ratio (iFR).

FIG. 8 illustrates a catheter similar to that described in FIG. 5 but equipped with a delivery device. The catheter 521 may have at least one distal aperture 503 and a pressure assessing port 520. The pressure assessing port may be coupled to an external pressure transducer. In this example, the catheter 521 has a delivery device that is a delivery catheter 551 with a balloon 553 at its distal end. The delivery catheter may have a port 552 at its proximal end. The port may be used to facilitate the delivery apparatus, such as inflating and deflating the balloon 553.

The catheter 521 and the delivery catheter 551 may be advanced into the blood vessel to the area of interest simultaneously. The catheter may be configured, as described in FIGS. 4 and 5, to hold a column of fluid to measure the pressure at the distal aperture 503 as the catheter is advanced through the blood vessel. The pressure transducer may collect the pressure measurements at the distal aperture 503. The combined pressure assessment catheter and delivery device may take real-time pressure measurements immediately prior to and after the interventional device is placed. For example, a stent may be placed over the balloon 553 and the catheter 521, and delivery catheter 551 may be advanced to a stent deployment site. Once the stent deployment site is reached, the distal aperture 503 may be located beyond the site and the balloon 553 and stent are within the site. The pressure transducer may collect pre-implantation pressure readings at the distal aperture 503. The user may inflate the balloon 553 by injecting a solution, such as saline, into the port 552 to the balloon 553. The balloon 553 may inflate to deploy the stent at the stent deployment site within the blood vessel. The user may deflate the balloon 553 by removing the solution through the port 552. Once normal flow through the now-stented region resumes, the pressure transducer may begin collecting post-implantation pressure measurements at the distal aperture 503. This will allow the user to verify the proper implantation of an interventional device in real-time. With these instantaneous pressure measurements, the operator may make decisions as to whether to readjust the interventional device. This will reduce the time of the procedure thus reducing potential health risks to the patient.

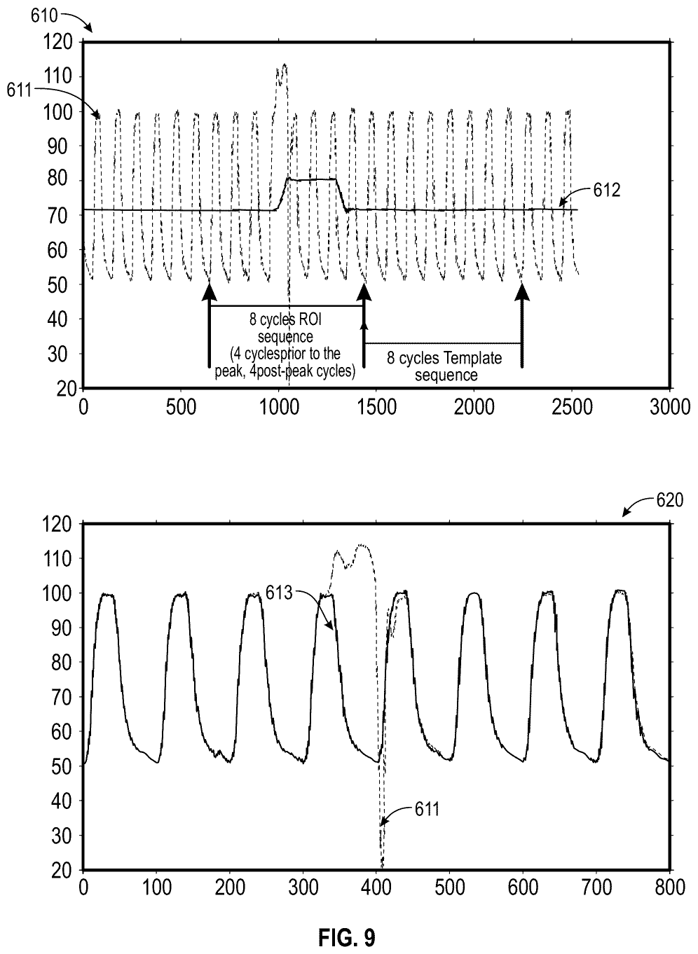

FIG. 9 illustrates the raw pressure measurements and the averaged pressure waveform. Distal pressure measurements collected by the intravascular instrument are processed within the subsystem. The pressure measurements from the intravascular instrument may be charted as depicted in 610. The pressure measurements are plotted on chart 610 in sequential order based on their timestamp or time interval. The raw pressure data 611 may be plotted with the cardiac cycle waveform 612. The representation of the raw pressure measurements 611 are used to create the averaged waveform 613.

One or more processors may determine the initial time the distal aperture reached the area of interest, or the region of interest (“ROI”), such as a stenosed area, by identifying a first spike in pressure reading of the collected plurality of pressure measurements as the intravascular instrument moves through the area of interest. Where the spike begins to drop, the distal aperture has passed beyond the stenosed area. The averaged waveform 613 are calculated using a specific number of cycles of measurements at the ROI. These cycles may include the measurements just prior to and just after the intravascular instrument reaches the ROI. For example, the 8 cycles to calculate the averaged waveform includes the peak from the ROI and 4 cycles prior to the peak and 4 cycles post peak.

The system may use the raw pressure measurements of the same length of the cycle used for the averaged waveform 613 from prior to or after the section with the ROI as a template sequence for comparison. For example, the 8 cycles including the ROI (“ROI sequence”) may be used to calculate the averaged waveform 613 and the 8 cycles following the ROI sequence may be used as template to calculate the average waveform 613. Once the averaged waveform 613 is determined, it may be plotted at depicted in 620, with the specific cycles of the raw pressure measurements 611 used to obtain the averaged waveform 613.

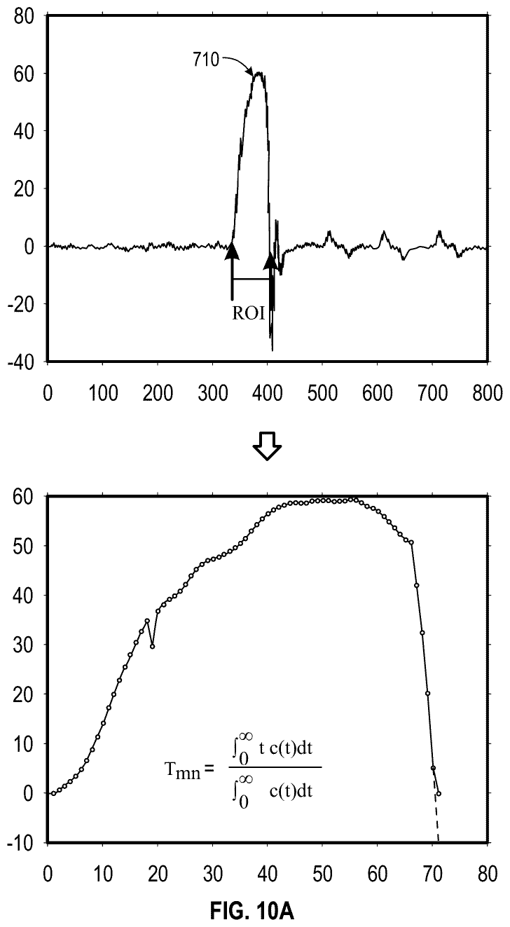

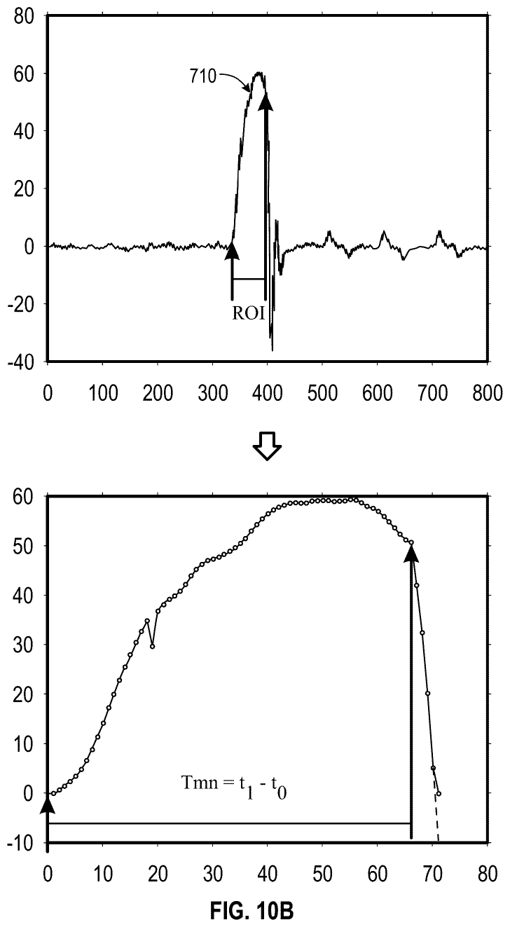

FIG. 10A illustrates an example pressure dilution curve. The pressure dilution curve 710 illustrates the pressure measurement at the intravascular instrument at a given time as the distal aperture advances through the blood vessel past the ROI. For example, pressure dilution curve 710 may chart the pressure measurement in inches of mercury (inHg) based on the time the measurement was taken. The initial time and the applied log normal fit may be used to determine the mean transit time. Time is on the x-axis of the pressure dilution curve.

The pressure dilution curve 710 may be used to determine the mean transit time “Tmn.” Mean transit time may be the flow rate of blood within the blood vessel.

The pressure dilution curve 710 may have a pressure measurement “p.” In some examples, the pressure measurement “p” of curve 710 may be defined by any of the plurality of pressure measurement between a first pressure measurement and a last pressure measurement in a plurality of pressure readings collected by the intravascular instrument as the pressure sensing catheter advances through the blood vessel. The pressure measurement “p” is depicted in FIG. 10A at the area marked by ROI.

The integral of curve 710 may be used to calculate the mean transit time “Tmn” of the blood vessel. The mean transit time “Tmn” may be determined using the initial time “tₒ” determined using pressure dilution curve 710 and the beginning point of pressure measurement “p”. For example, the mean transit time may be determined using a ratio that is a function of time multiplied by the average pressure at the distal aperture within the blood vessel at the given time to the average pressure at the distal aperture within the blood vessel. According to some examples, the mean transit time “Tmn” may be determined using the following equation:

$${T}_{mn} = \frac{{\int }_{0}^{∞} t \cdot c ( t ) \cdot dt}{{\int }_{0}^{∞} c ( t ) \cdot dt}$$

In the equation, “t” may correspond to time. The time may be the time-stamped time corresponding to any given pressure measurement. The “c” in the equation may correspond to the average pressure at the distal aperture at that time “t.”

FIG. 10B illustrates another example pressure dilution curve. The pressure dilution curve 710 illustrates the pressure measurement collected by the intravascular instrument at a given time as the distal aperture advances through the blood vessel past the ROI. For example, pressure dilution curve 710 may chart the pressure measurement in inches of mercury (inHg) based on the time the measurement was taken. The initial time and the applied log normal fit may be used to determine the mean transit time. Time is on the x-axis of the pressure dilution curve.

The pressure dilution curve 710 may be used to determine the mean transit time “Tmn.” Mean transit time may be the flow rate of blood within the blood vessel.

The pressure dilution curve 710 may have a pressure measurement “p.” In some examples, the pressure measurement “p” of curve 710 may be defined by any of the plurality of pressure measurement between a first pressure measurement where the distal aperture reaches the ROI and a point where the pressure readings begin to rapidly drop. The pressure measurement “p” is depicted in FIG. 10B at the area marked by ROI.

The integral of curve 710 may be used to calculate the mean transit time “Tmn” of the blood vessel. The mean transit time “Tmn” may be determined using the initial time “tₒ” determined using pressure dilution curve 710 and the dropping point of pressure measurement “p”. For example, the mean transit time may be determined by subtracting the initial time “tₒ” from the time the pressure readings began to drop. According to some examples, the mean transit time “Tmn” may be determined using the following equation:

$${T}_{mn} = {t}_{1} - {t}_{0}$$

The mean transit time may be determined at rest and at hyperemia. Determining the mean transit time at rest may include collecting one or more pressure measurements when the distal aperture is held distal to the area of interest in a blood vessel in a natural state. For example, a natural state may be the state the blood vessel without any treatments or medications. Determining the mean transit time at hyperemia may include collecting one or more pressure measurements when the distal aperture is distal to the area of interest in a blood vessel that has been pharmacologically induced hyperemia to create full dilation of the blood vessel.

Under the current standard, the user will use temperature readings from a pressure wire to determine mean transit time. Specifically, the temperature sensor of the pressure wire to capture the thermodilution curve to determine the mean transit time. The thermodilution curve is bounded by the points in which the temperature rises and falls as a chilled bolus passes through the stenosed area or ROI.

The CFR may be determined using the determined mean transit time at rest “Tmn at rest” and the mean transit time during hyperemia “Tmn at hyperemia,” as shown in the below equation:

$$CFR = \frac{{T}_{mn at rest}}{{T}_{mn at hyperemia}}$$

According to some examples, the mean transit time at hyperemia “Tmn at hyperemia” may be used to determine the IMR. For example, IMR may be determined using the following equation:

$$IMR = {P}_{d at hyperemia} \times {T}_{mn at hyperemia}$$

In the equation “P_{d} at hyperemia” may correspond to the distal pressure during pharmacologically induced hyperemia. The distal pressure may be determined based on the intravascular data collected by the intravascular instrument. For example, the pressure measurements collected by the intravascular instrument when located distal to the area of interest may be used as “P_{d at hyperemia}.” Based on Ohm's law, the pressure gradient (ΔP) may be equal to the flow rate (Q) multiplied by the resistance of the vessel (R), as shown in the equation below:

$$Δ P = QR$$

Flow rate may be calculated by theoretical aortic and venous pressures through the resistor model.

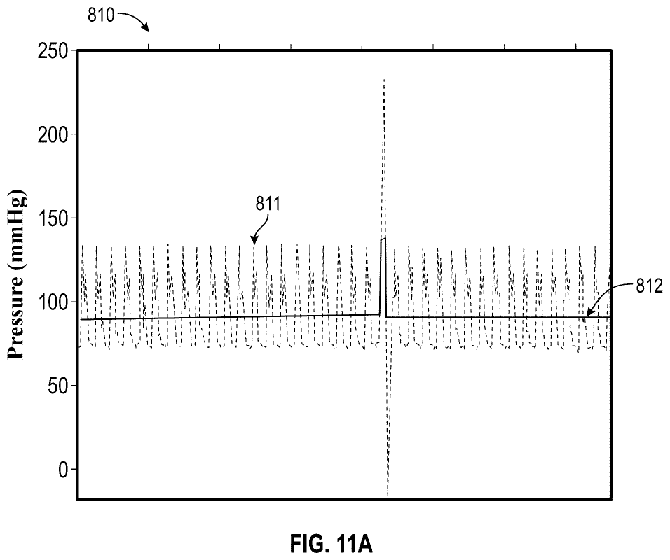

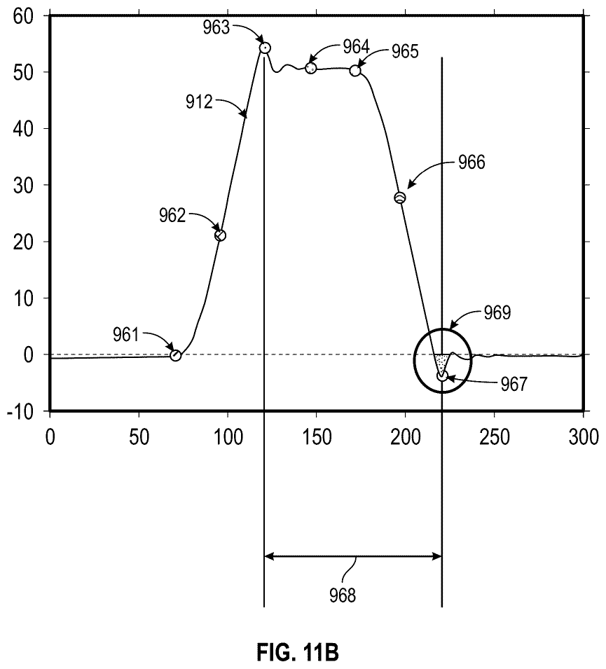

FIGS. 11A-B illustrate another method of calculating the mean transit time of the blood vessel. FIG. 11A, similar to chart 610 of FIG. 9, illustrates the pressure measurements from an intravascular instrument plotted in chart 810, wherein the pressure measurements are measured in millimeters of mercury (mmHg) by time in milliseconds (ms). The pressure measurements are plotted on chart 810 in sequential order based on their timestamp or time interval. In some examples, there may be a moving average filter applied to the pressure measurements from the intravascular instrument, depicted as raw pressure waveform 811. In some examples, the window length of chart 810 may be equivalent to one cardiac cycle. A filtered waveform 812 may be generated and overlaid onto the raw pressure waveform 811 in chart 810, similar to chart 610 in FIG. 9.

FIG. 11B depicts further processing of the filtered waveform 912. The filtered waveform 912 may be processed by a subsystem. The subsystem may be similar to subsystem 108 described in FIG. 1. The subsystem may be communicatively coupled to an intravascular tool with pressure sensing capabilities, such as a pressure wire or catheter. Pressure readings from the intravascular instrument with pressure sensing capabilities may be transmitted to the subsystem.

In some examples, the subsystem may extract various landmarks along filtered waveform 912 to assist in processing, analyzing data, and determining characteristics of the blood vessel, such as the mean transit time. The various landmarks may be extracted based on manual settings or filters built into the subsystem. In some examples, the various landmarks may be automatically extracted based on prior data stored by the system or subsystem. The various landmarks in filtered waveform 912 may include a zero anchoring point 961, an ascending point 962, a highest peak 963, a stabilized point 964, a drop-off point 965, a descending point 966, and a negative peak 967. Filtered waveform 912 is aligned with the zero on the y-axis based on anchoring point 961.

Filtered waveform 912 may be used to calculate the mean transit time of the blood vessel. In some examples, negative peak 967 falls below the zero on the y-axis, creating a defined area 969 under the curve. In FIG. 11B, defined area 969 is depicted as the shaded area below the zero on the y-axis. Further, the time between highest peak 963 and negative peak 967 is depicted as the change in time 968. The mean transit time may be calculated by multiplying the area of defined area 969 by change in time 968. By including change in time 968, the calculated mean transit time is adjusted by the heart rate of a patient. According to some examples, the mean transit time “Tmn” may be calculated using the following equation:

$${T}_{mn} = a \times b$$

In the equation, “a” may correspond to the area under the curve below the zero of the y-axis, described above as defined area 969. The “b” in the equation may correspond to the amount of time between highest peak 963 and negative peak 967, described above as change in time 968.

FIGS. 12A-C illustrate a comparison of the results of determining the mean transit time using thermodilution and the area-under-the-curve (“AUC”) method. FIG. 12A shows the true flow rate by the thermodilution mean transit time. The true flow rate and thermodilution mean transit time were measured and collected by utilizing an inline flow meter and a pressurewire, accordingly, to serve as the study controls, representing an industry standard for calculating mean transit time. The data represented in FIG. 12A are from 122 samples with various combinations of different pressures, flow rates, and heart rates.

FIG. 12B shows the correlation between the AUC mean transit time and the true flow rate. The correlation coefficient between the AUC mean transit time and the true flow rate is −0.77. FIG. 12C shows the correlation between the AUC mean transit time and the thermodilution mean transit time. The correlation coefficient between the AUC mean transit time and the thermodilution mean transit time is 0.7. The correlation coefficients may be a statistical measure that quantifies the strength and direction of a linear relationship between two variables, wherein above 0.7 or below −0.7 may be considered a strong correlation.

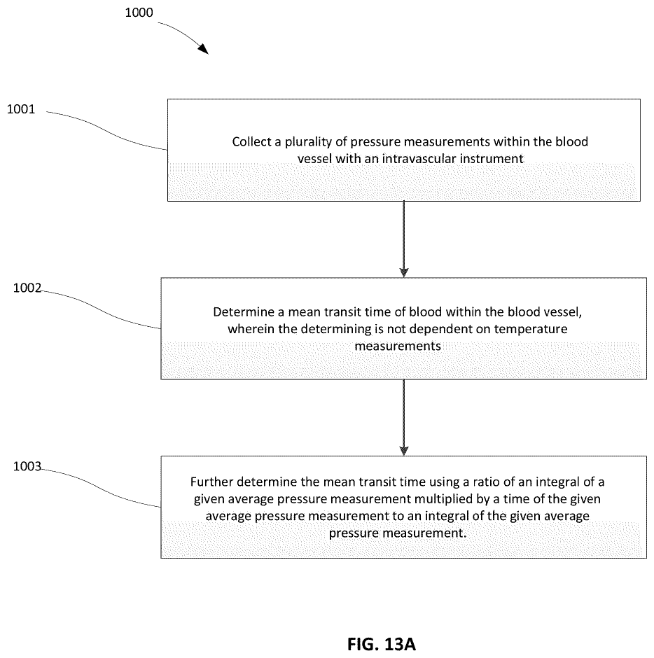

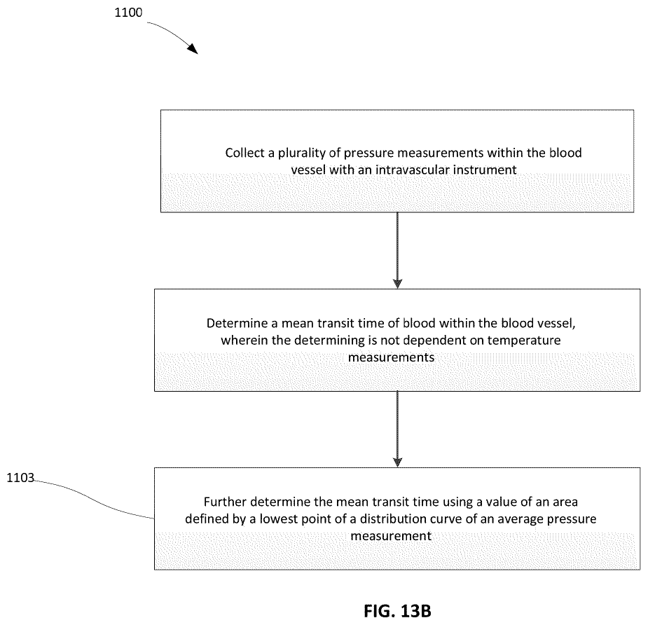

FIG. 13A illustrates example method 1000 of determining a mean transit time of the blood within a blood vessel. The following operations do not have to be performed in the precise order described below. Rather, various operations can be handled in a different order or simultaneously, and operations may be added or omitted.

In block 1001, a plurality of pressure measurements may be received. For example, a data collection system, such as system 100, may collect a plurality of pressure measurements using the intravascular instrument. The pressure measurements may be collected by a pressure wire, a probe or a catheter having one or more distal apertures. The pressure measurements may be communicated to the subsystem via the receiver. The pressure measurements are stored in memory 114. One or more of the processors may determine the average pressure measurement based on the plurality of pressure measurements. Further, one or more of the processors may create a distribution curve based on the average pressure measurements.

In block 1002, the mean transit time of the blood within the blood vessel may be determined without temperature readings. Using the distribution curve, one or more of the processors may determine the mean transit time of the blood within the blood vessel. One or more of the processors may integrate the distribution curve when determining the mean transit time of the blood within the blood vessel. One or more of the processors may be configured to use the determined mean transit time to determine at least one of a CFR value or an IMR value.

In block 1003, the mean transit time may be further determined using a ratio of an integral of a given average pressure measurement multiplied by a time of the given average pressure measurement to an integral of the given average pressure measurement.

FIG. 13B illustrates an example method 1100 of determining a mean transit time of the blood within a blood vessel. The following operations do not have to be performed in the precise order described below. Rather, various operations can be handled in a different order or simultaneously, and operations may be added or omitted. Method 1100 may contain many of the same features as method 1000 described in FIG. 13A.

Similar to method 1000, method 1100 may use a distribution curve of pressure readings to determine the mean transit time of blood within the blood vessel, wherein the determining is not dependent on temperature measurements. In block 1103, the mean transit time may be further determined using a value of an area defined by a lowest point of a distribution curve of an average pressure measurement. The distribution curve may be based on pressure measurements collected by an intravascular instrument inserted in the blood vessel of a patient. In some examples, the distribution curve may be derived from raw pressure measurements plotted in sequential order based on their time stamp. The raw pressure measurements may be processed into the distribution curve using a moving average filter. The distribution curve may be the same as and undergo processing similar to that described of filtered waveform 912 in FIGS. 11A-B.

In some examples, the mean transit time may be determined using the area bounded by the lowest point of the distribution curve and the zero of the y-axis. The mean transit time may further be determined using the change in time between the highest point and the lowest point of the distribution curve.

The disclosed methods and apparatus will allow for a simpler procedure allowing for more consistent results and less time spent obtaining the results. Methods that use temperature measurements to obtain the mean transit time require the bolus to be chilled to a certain temperature prior to injecting it into the blood vessel. These methods require that the operator is highly skilled and works hastily with the chilled material before the temperature drops. These methods are prone to mistakes and potentially call for multiple tries to collect accurate readings. The disclosed method avoids these issues by allowing the mean transit time to be determined without the use of temperatures, thus reducing procedure time and therefore increasing the safety of the patient. Moreover, by determining the mean transit time without the use of temperature measurements, the process of determining mean transit time is more cost-effective than other methods that require the use of electrical sensors in the blood vessel to enable temperature measurements.

According to some examples, at least one of a CFR value or an IMR may be determined based on the determined mean transit time. For example, the CFR value may be determined by dividing the determined mean transit time at rest by the mean transit time during hyperemia. The IMR value may be determined by multiplying the mean transit time at hyperemia by the distal pressure during hyperemia.

The determined CFR value and/or IMR may be used in conjunction with one or more patient factors when determining microvascular disease and potential treatments. For example, a physician may consider the patient's age, gender, BMI, medical history, the vessel type, the vessel condition, prior treatments, etc. in conjunction with the determined CFR and/or IMR value to determine if there is any microvascular disease and/or a potential treatment.

In one example, microvascular resistance may be determined based on the determined mean transit time, the CFR value and/or the IMR value, and the age of the patient. For example, the age of the patient may be input and processed in conjunction with the CFR value and/or IMR value to determine the microvascular resistance of the blood vessel. According to some examples, one or more age specific parameters may be introduced based on training data. In some examples, the age specific parameters may be ranges of age to categorize the patient. The training data may be collected and used as input into a machine learning model. Based on the one or more age specific parameters as well as the mean transit time, the CFR value, and/or the IMR value, the machine learning model may determine the microvascular resistance of the blood vessel.

In one example, microvascular resistance may be determined based on the determined mean transit time, the CFR value and/or the IMR value, and the gender of the patient. For example, the gender of the patient may be input and processed in conjunction with the CFR value and/or IMR value to determine the microvascular resistance of the blood vessel. In some examples, the mean transit time, the CFR value, and/or the IMR value may be different based on the patient's gender. The patient's gender may be introduced based on training data. The training data may be collected and used as input into a machine learning model. Based on the patient's gender as well as the mean transit time, the CFR value, and/or the IMR value, the machine learning model may determine the microvascular resistance of the blood vessel.

In one example, microvascular resistance may be determined based on the determined mean transit time, the CFR value and/or the IMR value, and the BMI of the patient. For example, the BMI of the patient may be input and processed in conjunction with the CFR value and/or IMR value to determine the microvascular resistance of the blood vessel. According to some examples, one or more BMI parameters may be introduced based on training data. In some examples, the BMI parameters may be ranges of age to categorize the patient. The training data may be collected and used as input into a machine learning model. Based on the one or more BMI parameters as well as the mean transit time, the CFR value, and/or the IMR value, the machine learning model may determine the microvascular resistance of the blood vessel.

In one example, microvascular resistance may be determined based on the determined mean transit time, the CFR value and/or the IMR value, and the medical history of the patient. By way of example, medical history, such as known heart failure, of the patient may be input and processed in conjunction with the CFR value and/or IMR value to determine the microvascular resistance of the blood vessel. According to some examples, one or more condition specific parameters may be introduced based on training data. In some examples, the condition specific parameters may be based on the medical history of the patient. For example, the medical history of the patient may indicate a history of heart failure. The training data may be collected and used as input into a machine learning model. Based on the one or more condition specific parameters as well as the mean transit time, the CFR value, and/or the IMR value, the machine learning model may determine the microvascular resistance of the blood vessel.

As another example, the medical history, such as known a previous diagnosis of diabetes, of the patient may be input and processed in conjunction with the CFR value and/or IMR value to determine the microvascular resistance of the blood vessel. According to some examples, one or more condition specific parameters may be introduced based on training data. In some examples, the condition specific parameters may be based on the medical history of the patient. For example, the medical history of the patient may indicate a history of diabetes. The training data may be collected and used as input into a machine learning model. Based on the one or more condition specific parameters as well as the mean transit time, the CFR value, and/or the IMR value, the machine learning model may determine the microvascular resistance of the blood vessel.

As another example, the medical history, such as known a previous diagnosis of hypertension, of the patient may be input and processed in conjunction with the CFR value and/or IMR value to determine the microvascular resistance of the blood vessel. According to some examples, one or more condition specific parameters may be introduced based on training data. In some examples, the condition specific parameters may be based on the medical history of the patient. For example, the medical history of the patient may indicate a history of hypertension. The training data may be collected and used as input into a machine learning model. Based on the one or more condition specific parameters as well as the mean transit time, the CFR value, and/or the IMR value, the machine learning model may determine the microvascular resistance of the blood vessel.

In one example, microvascular resistance may be determined based on the determined mean transit time, the CFR value and/or the IMR value, and the vessel type being diagnosed. By way of example, vessel type, such as the left anterior descending (“LAD”) artery, of the patient may be input and processed in conjunction with the CFR value and/or IMR value to determine the microvascular resistance of the blood vessel. According to some examples, one or more vessel specific parameters may be introduced based on training data. In some examples, the vessel specific parameters may be based on the vessel type. For example, the type may be a LAD. The training data may be collected and used as input into a machine learning model. Based on the one or more vessel specific parameters as well as the mean transit time, the CFR value, and/or the IMR value, the machine learning model may determine the microvascular resistance of the blood vessel.

As another example, vessel type, such as left circumflex artery (“LCX”), of the patient may be input and processed in conjunction with the CFR value and/or IMR value to determine the microvascular resistance of the blood vessel. According to some examples, one or more vessel specific parameters may be introduced based on training data. In some examples, the vessel specific parameters may be based on the vessel type. For example, the type may be an LCX. The training data may be collected and used as input into a machine learning model. Based on the one or more vessel specific parameters as well as the mean transit time, the CFR value, and/or the IMR value, the machine learning model may determine the microvascular resistance of the blood vessel.

As another example, vessel type, such as the right coronary artery (“RCA”), of the patient may be input and processed in conjunction with the CFR value and/or IMR value to determine the microvascular resistance of the blood vessel. According to some examples, one or more vessel specific parameters may be introduced based on training data. In some examples, the vessel specific parameters may be based on the vessel type. For example, the type may be an RCA. The training data may be collected and used as input into a machine learning model. Based on the one or more vessel specific parameters as well as the mean transit time, the CFR value, and/or the IMR value, the machine learning model may determine the microvascular resistance of the blood vessel.

As another example, vessel type, such as the left marginal artery, of the patient may be input and processed in conjunction with the CFR value and/or IMR value to determine the microvascular resistance of the blood vessel. According to some examples, one or more vessel specific parameters may be introduced based on training data. In some examples, the vessel specific parameters may be based on the vessel type. For example, the type may be a left marginal artery. The training data may be collected and used as input into a machine learning model. Based on the one or more vessel specific parameters as well as the mean transit time, the CFR value, and/or the IMR value, the machine learning model may determine the microvascular resistance of the blood vessel.

As another example, vessel type, such as diagonal arteries, of the patient may be input and processed in conjunction with the CFR value and/or IMR value to determine the microvascular resistance of the blood vessel. According to some examples, one or more vessel specific parameters may be introduced based on training data. In some examples, the vessel specific parameters may be based on the vessel type. For example, the type may be diagonal arteries. The training data may be collected and used as input into a machine learning model. Based on the one or more vessel specific parameters as well as the mean transit time, the CFR value, and/or the IMR value, the machine learning model may determine the microvascular resistance of the blood vessel.

As another example, vessel type, such as the right marginal artery, of the patient may be input and processed in conjunction with the CFR value and/or IMR value to determine the microvascular resistance of the blood vessel. According to some examples, one or more vessel specific parameters may be introduced based on training data. In some examples, the vessel specific parameters may be based on the vessel type. For example, the type may be a right marginal artery. The training data may be collected and used as input into a machine learning model. Based on the one or more vessel specific parameters as well as the mean transit time, the CFR value, and/or the IMR value, the machine learning model may determine the microvascular resistance of the blood vessel.

In one example, microvascular resistance may be determined based on the determined mean transit time, the CFR value and/or the IMR value, and patient treatment history. By way of example patient treatment history, such as prior percutaneous coronary intervention (“PCI”), may be input and processed in conjunction with the CFR value and/or IMR value to determine the microvascular resistance of the blood vessel. According to some examples, one or more treatment parameters may be introduced based on training data. In some examples, the treatment parameters may be based on a patient's treatment history. For example, the patient may have had a prior PCI. Prior PCI may generate microvascular damage and, therefore, may be considered when determining the microvascular resistance of the blood vessel. The training data may be collected and used as input into a machine learning model. Based on the one or more vessel specific parameters as well as the mean transit time, the CFR value, and/or the IMR value, the machine learning model may determine the microvascular resistance of the blood vessel.

As another example, patient treatment history, such as coronary artery bypass graft (“CABG”), may be input and processed in conjunction with the CFR value and/or IMR value to determine the microvascular resistance of the blood vessel. According to some examples, one or more treatment parameters may be introduced based on training data. In some examples, the treatment parameters may be based on a patient's treatment history. For example, the patient may have had a prior CABG. CABG may alter the cardiac physiology and/or may lead to microvascular alternations and, therefore may be considered when determining the microvascular resistance of the blood vessel. The training data may be collected and used as input into a machine learning model. Based on the one or more vessel specific parameters as well as the mean transit time, the CFR value, and/or the IMR value, the machine learning model may determine the microvascular resistance of the blood vessel.

The aspects, examples, features, and examples of the disclosure are to be considered illustrative in all respects and are not intended to limit the disclosure, the scope of which is defined only by the claims. Other examples, modifications, and usages will be apparent to those skilled in the art without departing from the spirit and scope of the claimed invention.

Throughout the application, where compositions are described as having, including, or comprising specific components, or where processes are described as having, including, or comprising specific process steps, it is contemplated that compositions of the present teachings also consist essentially of, or consist of, the recited components, and that the processes of the present teachings also consist essentially of, or consist of, the recited process steps.

In the application, where an element or component is said to be included in and/or selected from a list of recited elements or components, it should be understood that the element or component can be any one of the recited elements or components and can be selected from a group consisting of two or more of the recited elements or components. Further, it should be understood that elements and/or features of a composition, an apparatus, or a method described herein can be combined in a variety of ways without departing from the spirit and scope of the present teachings, whether explicit or implicit herein.

The use of the terms “include,” “includes,” “including,” “have,” “has,” or “having” should be generally understood as open-ended and non-limiting unless specifically stated otherwise.

The use of the singular herein includes the plural (and vice versa) unless specifically stated otherwise. Moreover, the singular forms “a,” “an,” and “the” include plural forms unless the context clearly dictates otherwise. In addition, where the use of the term “about” is before a quantitative value, the present teachings also include the specific quantitative value itself, unless specifically stated otherwise. As used herein, the term “about” refers to a ±10% variation from the nominal value. All numerical values and ranges disclosed herein are deemed to include “about” before each value.

It should be understood that the order of steps or order for performing certain actions is immaterial so long as the present teachings remain operable. Moreover, two or more steps or actions may be conducted simultaneously.

Where a range or list of values is provided, each intervening value between the upper and lower limits of that range or list of values is individually contemplated and is encompassed within the invention as if each value were specifically enumerated herein. In addition, smaller ranges between and including the upper and lower limits of a given range are contemplated and encompassed within the invention. The listing of exemplary values or ranges is not a disclaimer of other values or ranges between and including the upper and lower limits of a given range.

Although the present disclosure has been described with reference to particular examples, it is to be understood that these examples are merely illustrative of example applications and implementations. It is therefore to be understood that numerous modifications may be made to the illustrative examples and that other arrangements may be devised without departing from the spirit and scope of the appended claims.

1. A method, comprising:
receiving, at one or more processors, intravascular data for a blood vessel, the intravascular data comprising a plurality of pressure measurements at a location within the blood vessel collected by an intravascular instrument; and
determining, by the one or more processors based on the plurality of pressure measurements, a mean transit time of blood within the blood vessel, wherein the determining is not dependent on temperature measurements.

2. (canceled)

3. The method of claim 1, further comprising:
determining, by the one or more processors based on the plurality of pressure measurements, an average pressure measurement;
creating, by the one or more processors based on the determined average pressure measurement, a distribution curve.

4. (canceled)

5. (canceled)

6. The method of claim 3, wherein determining the mean transmit time is further based on the distribution curve.

7. The method of claim 3, wherein determining the mean transit time further includes determining, by the one or more processors, a ratio of an integral of a given average pressure measurement multiplied by a time of the given average pressure measurement to an integral of the given average pressure measurement.

8. The method of claim 6, wherein the one or more processors are further configured to determine, based on the determined mean transit time, at least one of a coronary flow reserve (“CFR”) value or an index of microcirculatory resistance (“IMR”) value.

9. The method of claim 3, wherein when determining the average pressure measurement, the one or more processors are further configured to use a plurality of cycles of the plurality of the intravascular data from including the pressure readings before during and after the intravascular instrument reaches a location in the blood vessel.

10. (canceled)

11. (canceled)

12. The method of claim 1, wherein the intravascular instrument has pressure sensing capabilities with a pressure transducer connected to a pressure assessing port.

13. The method of claim 3, further comprising
determining the averaged pressure waveform by identifying a first pressure measurement of the plurality of pressure measurements as the intravascular instrument advances to the area of interest.

14. A system comprising:
one or more processors configured to:
receive intravascular data for a blood vessel, the intravascular data comprising a plurality of pressure measurements at a location within the blood vessel; and
determine, by the one or more processors based on the plurality of pressure measurements, a mean transit time of blood within the blood vessel, wherein the determining is not dependent on temperature measurements.

15-24. (canceled)

25. A pressure sensing catheter comprising:
a hypotube configured to deliver a hemocompatible fluid to a location of a blood vessel;
an outer member having a proximal end and a distal end, wherein the proximal end is secured to the hypotube;
an inner member having a proximal end and a distal end, wherein the proximal end of the inner member is secured to the outer member; and
at least one aperture on the distal end of the outer member, the aperture allowing for measurement of pressure at the location within the blood vessel.

26. A pressure sensing catheter as claimed in claim 25 wherein
one or more processors coupled to the pressure sensing catheter configured to:
receive pressure measurements; and
determine, by the one or more processors based on the plurality of pressure measurements, a mean transit time of blood within the blood vessel, wherein the determining is not dependent on temperature measurements.

27. The pressure sensing catheter of claim 26, further comprising a pressure transducer in fluid communication with at least one of the apertures on the distal end of the outer member.

28. The pressure sensing catheter of claim 25, wherein the one or more processors are further configured to determine, based on the determined mean transit time, at least one of a CFR value or an IMR value.

29. The pressure sensing catheter of claim 25, wherein the one or more processors are further configured to:
determine average pressure measurements; and
create, based on the determined average pressure measurements, a distribution curve.

30. The pressure sensing catheter of claim 29, wherein determining the mean transit time of the blood within the blood vessel is further based on the distribution curve.

31. The pressure sensing catheter of claim 26, wherein determining the mean transit time further includes determining, by the one or more processors, a ratio of an integral of a given average pressure measurement multiplied by a time of the given average pressure measurement to an integral of the given average pressure measurement.

32. The pressure sensing catheter of claim 26, wherein determining the average pressure measurement further includes using, by the one or more processors, a plurality of cycles of the plurality of the intravascular data from including the pressure readings before during and after the intravascular instrument reaches a location in the blood vessel.

33. The pressure sensing catheter of claim 26, wherein, when determining the mean transit time, the one or more processors are further configured to integrate the distribution curve.

34. The pressure sensing catheter of claim 25, wherein the plurality of pressure measurements is collected distal to an area of interest in the blood vessel.

35. The method of claim 3, wherein the mean transit time is determined using a value of an area defined by the lowest point of the distribution curve.
